(19) United States
(12) Patent Application Publication
(10) Pub. No.: US 2023/0323325 A1
(43) Pub. Date: Oct. 12, 2023

(54) PROGRAMMABLE DELIVERY OF RNA-GUIDED CRISPR-CAS PROTEINS TO SUBCELLULAR ORGANELLES

(71) Applicant: MONTANA STATE UNIVERSITY, Bozeman, MT (US)

(72) Inventors: Blake A. WIEDENHEFT, Bozeman, MT (US); Anna A. NEMUDRAIA, Bozeman, MT (US); Artem A. NEMUDRYI, Bozeman, MT (US)

(73) Assignee: MONTANA STATE UNIVERSITY, Bozeman, MT (US)

(21) Appl. No.: 18/299,692

(22) Filed: Apr. 12, 2023

Related U.S. Application Data
(60) Provisional application No. 63/329,952, filed on Apr. 12, 2022.
(60) Provisional application No. 63/450,339, filed on Mar. 6, 2023.

(51) Int. Cl. C12N 9/22; C12N 15/11; C12N 15/86

(52) U.S. Cl. CPC C12N 9/22; C12N 15/11; C12N 15/86; C12N 2310/20

(57) ABSTRACT

Engineered CRISPR proteins may be generated for localized anchoring to targeted cellular locations. Engineered CRISPR proteins may be generated by a lipidation motif with a CRISPR protein. The lipidation motif may be post-translationally modified to anchor the lipidation motifs and the fused CRISPR protein to a targeted cellular location, such as membranes of organelles associated with viral infections or other ailments. To account for possible additional amino acids that might affect the efficiency of post-translational modifications, linkers derived from C-terminal ends OAS1 (p46 isoform) and ZAP-L proteins may be used. Different fusion designs and/or different lipidation motifs may be used to target CRISPR proteins to specific and respective cellular locations. Targeted cellular localization of engineered CRISPR proteins may enable targeted therapies involving the engineered CRISPR proteins.

### CROSS-REFERENCE TO RELATED APPLICATIONS

This application claims the benefit of priority of U.S. Provisional Application No. 63/329,952, filed on Apr. 12, 2022, and U.S. Provisional Application No. 63/450,339, filed on Mar. 6, 2023, which are each incorporated by reference in their entireties herein for all purposes.

### GOVERNMENT LICENSE RIGHTS

This invention was made with government support under contract R35GM134867 awarded by the National Institutes of Health. The government has certain rights in the invention.

### BACKGROUND

RNA viruses (e.g., Coronaviruses, Flaviviridae, Filoviridae, Orthomyxoviridae, etc.) pose a global threat for human, plant, and animal health. Viral infections are often resistant to existing drugs and there is a critical need for innovative therapeutics that are responsive to the emergence of new viral variants, seasonal outbreaks, or novel viral pandemics. RNA-targeting CRISPR nucleases have the potential to be developed as effective, programmable, and versatile antiviral treatments that also have considerable value for testing gene function during drug development for the design of attenuated genotypes for vaccine development. However, many RNA viruses have evolved strategies to protect their RNA from cytosolic RNA-sensors and ribonucleases by sequestering viral RNA in replication organelles (ROs) that are composed of membranes sourced from specific organelles of the host. For example, replication organelles formed by flavi-, corona- and picornaviruses use membranes from the endoplasmic reticulum (ER) or Golgi apparatus, alphaviruses use plasma- and endo-lysosomal membranes, and nodaviruses use mitochondrial membranes.

### SUMMARY

The disclosure relates to techniques and systems for specific delivery of RNA-targeting CRISPR-Cas proteins and other effectors to cellular location. The cellular location may be subcellular locations such as viral replication organelles within infected cells of a subject. This delivery approach is generalizable to any protein or ribonucleoprotein complex.

In some aspects, the techniques described herein relate to a method of programmable delivery of Clustered Regularly Interspaced Short Palindromic Repeats (CRISPR)-Cas proteins for subcellular localization, the method including: fusing one or more lipidation motifs to a CRISPR-Cas protein to generate an engineered CRISPR protein, wherein the one or more lipidation motifs are post-translationally modified to anchor the one or more lipidation motifs and the fused CRISPR-Cas protein to a target cellular location; and administering the engineered CRISPR protein to a subject.

In some aspects, the techniques described herein relate to a method, wherein the cellular location is a membrane of a subcellular organelle.

In some aspects, the techniques described herein relate to a method, wherein the CRISPR-Cas protein is a CRISPR-Cas nuclease that cleaves target nucleic acid as part of a treatment regimen for the subject.

In some aspects, the techniques described herein relate to a method, wherein the target nucleic acid is viral nucleic acid.

In some aspects, the techniques described herein relate to a method, wherein the cellular location reduces cellular toxicity of the CRISPR-Cas nuclease.

In some aspects, the techniques described herein relate to a method, wherein the one or more lipidation motifs include a CTIL motif.

In some aspects, the techniques described herein relate to a method, wherein the one or more lipidation motifs include a CVIS motif.

In some aspects, the techniques described herein relate to a method, wherein fusing the one or more lipidation motifs to the CRISPR-Cas protein includes inserting a linker to account for amino acids that affect the efficiency of the post-translational modification.

In some aspects, the techniques described herein relate to a method, wherein the linker is derived from a C-terminal end of OAS1 or ZAP-L proteins.

In some aspects, the techniques described herein relate to an engineered Clustered Regularly Interspaced Short Palindromic Repeats (CRISPR)-Cas protein, including: a CRISPR-Case protein; and one or more lipidation motifs fused to the CRISPR-Cas protein, the one or more lipidation motifs being post-translationally modified to anchor the one or more lipidation motifs and the fused CRISPR-Cas protein to a target cellular location.

In some aspects, the techniques described herein relate to the engineered CRISPR-Cas protein, further including: a linker to account for amino acids that affect the efficiency of the post-translational modification.

In some aspects, the techniques described herein relate to a method of generating an engineered Clustered Regularly Interspaced Short Palindromic Repeats (CRISPR)-Cas protein, including: generating a library of CRISPR-Cas proteins including a CRISPR-Cas protein; and contacting one or more lipidation motifs with the library CRISPR-Cas proteins to fuse the one or more of lipidation motifs to the CRISPR-Cas protein, wherein the one or more lipidation motifs are post-translationally modified to anchor the one or more lipidation motifs and the fused CRISPR-Cas protein to a target cellular location.

### BRIEF DESCRIPTION OF THE FIGURES

Features of the present disclosure may be illustrated by way of example and not limited in the following figure(s), in which like numerals indicate like elements, in which:

FIG. 1 illustrates a schematic overview of targeting post-translation modified CRISPR nucleases to ROs;

FIG. 2 illustrates a schematic representation of fusion between CRISPR-associated (Cas) proteins and guide RNA (gRNA) ribonucleoprotein (RNP) complexes;

FIG. 3 is an image of a Western blot of protein expression of Cas-CAAX fusions in HEK 293T cells was tested using Western Blot with anti-HA-tag antibodies. ACTB is used as a loading control;

FIG. 4 illustrates an image of fluorescent microscopy, demonstrating that proteins-of-interest fused to lipidation motifs change subcellular localization in HEK 293T cells;

FIG. 5A illustrates a schematic diagram showing RNA editing with type-III CRISPR systems and splinted ligation;

FIG. 5B illustrates a schematic diagram of SthCsm-mediated cleavage;

FIG. 5C illustrates a schematic diagram showing examples of splint designs;

FIG. 5D illustrates a plot of a comparison of RNA ligation efficiency;

FIG. 5E illustrates plots of deep sequencing of RNA ligated using splint designs shown in FIG. 5C. Horizontal black bars in each of the plots show target site of the SthCsm complex;

FIG. 5F illustrates plots of quantification of editing outcomes shown in FIG. 5E;

FIG. 6A illustrates a diagram showing a pipeline for deleting 18 nucleotides in the GFP open reading frame (ORF) in the recombinant Sindbis-GFP (SINV-GFP) virus;

FIG. 6B illustrates a plot of RNA aliquots collected after the cleavage with Csm complex;

FIG. 6C illustrates a plot of RNA aliquots collected after and ligation with T4 RNA ligase;

FIG. 6D illustrates a melting curve analysis of the qPCR products in FIG. 6C;

FIG. 6E illustrates images of BHK-21 cells that were transfected with edited RNA of the SINV-GFP;

FIG. 6F illustrates melting curve analysis of the RT-qPCR products that were generated with RNA extracted from supernatants of BHK-21 cells 24 hours post transfection;

FIG. 7A illustrates a diagram showing a pipeline for substituting 18 nucleotides in the BFP open reading frame (ORF) in the recombinant Sindbis-BFP (SINV-BFP) virus;

FIG. 7B illustrates images of BHK-21 cells transfected with the edited viral RNA;

FIG. 7C illustrates images of transfected BHK-21 cells shown in FIG. 7B imaged with fluorescent microscope at 20× magnification;

FIG. 8A illustrates imaging of genomic RNA of Sindbis-GFP virus (SINV-GFP), which was edited to delete codons encoding for the chromophore (T65, Y66, G67) in the GFP gene;

FIG. 8B illustrates images of viral clones (n=36) that were collected from plaques and transferred into 48-well plate with BHK-21 cells;

FIG. 8C illustrates a schematic diagram of an SINV-GFP genome;

FIG. 8D illustrates a plot of frequency of programmed edits in the bulk virus population before the plaque purification; and

FIG. 8E illustrates a representative sequencing depth plot for a plaque-purified viral clone in FIG. 8B.

### DETAILED DESCRIPTION

Disclosed herein are techniques and systems for specific delivery of RNA-targeting CRISPR-Cas proteins and other effectors to viral replication organelles within infected cells. This delivery approach is generalizable to any protein or ribonucleoprotein complex.

Use of Membrane Targeting to Increase Efficacy of CRISPR-Based Antivirals

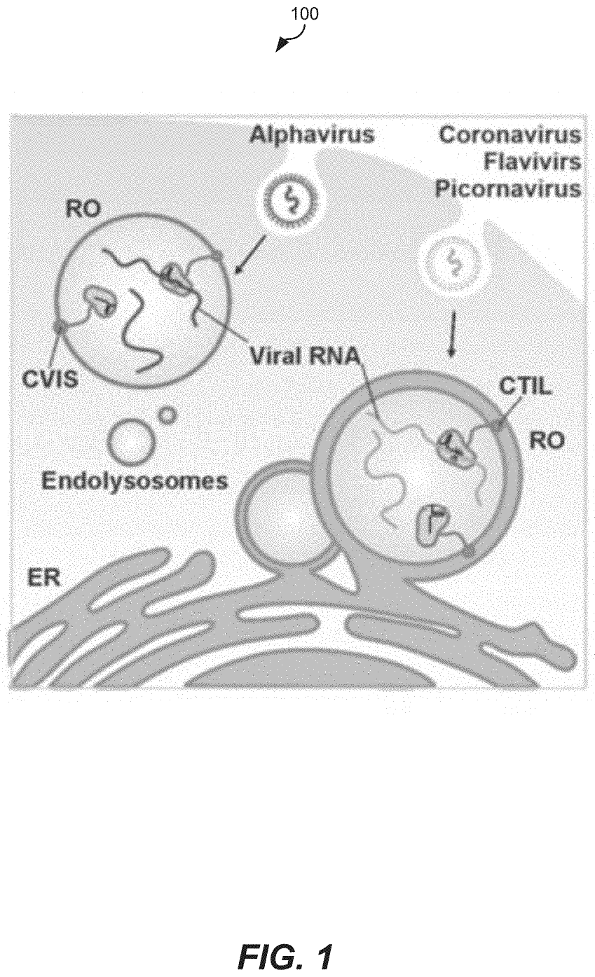

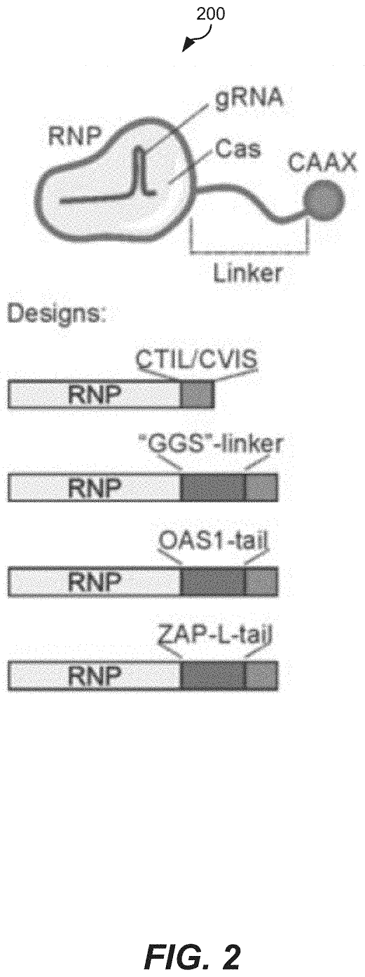

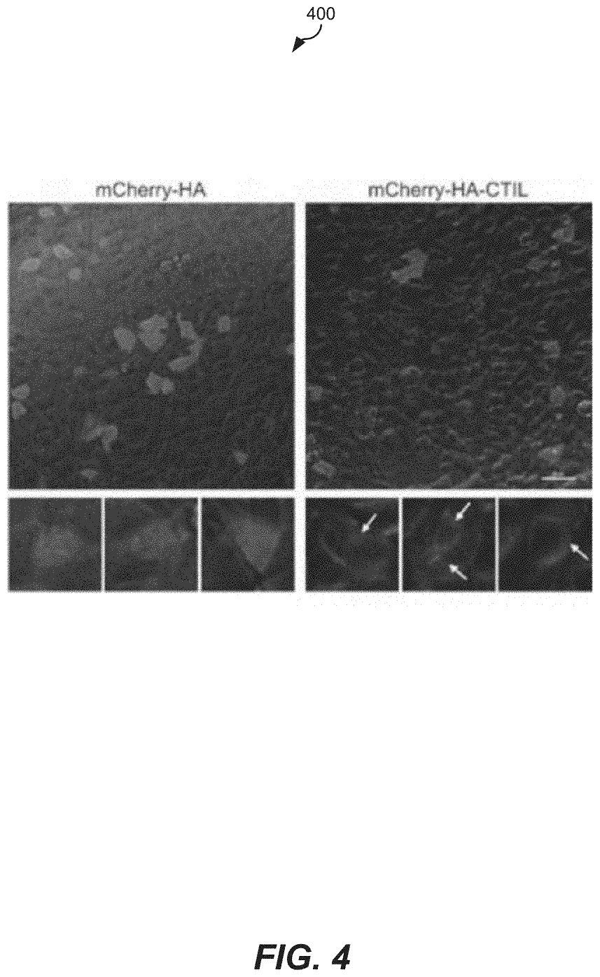

A subset of human antiviral proteins are targeted to cellular membranes and can access viral RNAs within the ROs. Specifically, RNA-sensors of the innate immunity, OAS1 and ZAP-L, encode “lipidation” motifs at the C-terminus (−CAAX motif) which are post-translationally modified to anchor proteins in the cellular membranes. Here, this natural mechanism is re-engineered by fusing lipidation motifs to CRISPR nucleases to anchor them in membranes appropriated by RNA viruses (as illustrated in FIG. 1). Different fusion designs and/or different lipidation motifs may be used to target CRISPR nucleases to specific cellular membranes associated with viral infections (as illustrated in FIG. 2). To account for possible additional amino acids that might affect the efficiency of post-translational modifications, linkers may be used. The linkers may be derived from C-terminal ends OAS1 (p46 isoform) and ZAP-L proteins (FIG. 1b). The CTIL motif in OAS1 facilitates targeting to ER membranes and is reported to be essential for antiviral activities of this protein against coronaviruses (i.e., SARS-CoV-2) and West Nile virus. Targeting of ZAP-L occurs via lipidation of a C-terminal CVIS motif, which is required for association with endo-lysosomal membranes and clearing infection with alphaviruses. Fusing CTIL motif to CRISPR nucleases may facilitate antiviral activities against flavi-, corona- and picornaviruses and other RNA viruses associated with ER membranes, while CVIS motif will be required for efficient targeting viruses associating with endo-lysosomal membranes (e.g., alphaviruses). A library of chimeric CRISPR nucleases is engineered by fusing these proteins to lipidation motifs and show that these proteins are efficiently expressed in human cells (illustrated in FIG. 4). Fluorescent microscopy is used to demonstrate that fusion of lipidation motifs changes subcellular localization (illustrated in FIG. 4).

Use of Membrane Targeting to Limit Cellular Toxicity of CRISPR Nucleases.

Type VI (i.e., Cas13) and type III (i.e., Cas7-11) CRISPR systems target RNA. To date, only Cas13 have been tested as antiviral tool in cell cultures and as a prophylactic drug in rodent models. Upon target recognition Cas13 activates a multi-turnover non-sequence-specific “collateral nuclease” activity, which can cause cell toxicity. Type III CRISPR systems rely on multisubunit CRISPR RNA (crRNA)-guided complexes that specifically cleave the complementary RNA at six nucleotide intervals. Recently described single protein Type III effector Cas7-11 cuts RNA without showing any collateral activity or cell toxicity. Targeting Cas13 to specific cellular membranes by design may enhance antiviral efficacy and deplete cytosolic levels of the protein, which may limit toxicity due to collateral damage to host RNAs. Compartmentalization of the nuclease may restrict collateral RNA damage to viral replication organelles and facilitates clearing of viral RNA. Alternatively, Cas7-11 nucleases lacking collateral activity may be used to limit nuclease activity to specific sites in viral RNAs.

Targeting Negative-Sense (−) RNA to Increase Efficacy of CRISPR-Based Antivirals.

Replication organelles are the production sites of viral RNAs that are further exported to cytoplasm for translation and packaging into new viral particles. Replication of (+) RNA viruses starts with producing a full-length negative-sense (−) RNA copy that is used as a template to replicate more viral genomes. While nascent (+) RNA copies are exported to cytoplasm, (−) RNA templates remain protected in the ROs. Destroying protected (−) RNA template rather than (+) RNA copies, or combination of both, may increase efficacy of CRISPR-based antivirals. Therefore, a combination of guide RNAs to target CRISPR nucleases fused to lipidation motifs to (−) RNA to efficiently eliminate viral RNA templates and to (+) RNA to degrade synthesized copies.

FIG. 1 illustrates a schematic overview 100 of targeting post-translation modified CRISPR nucleases to ROs. Lipidation at C-terminal motifs (e.g., −CAAX) anchors nucleases to the host membranes which are used by the viruses to form ROs. Prenylated “CTIL”-motif targets proteins predominantly to endoplasmic reticulum membranes, while prenylated “CVIS”—to endo-lysosomal and plasma membranes. illustrates Cas-effectors fused to prenylation motifs (−CAAX) are localized in replication organelles (ROs) for programmable targeting of viral RNAs. a Schematic overview of targeting post-translation modified CRISPR nucleases to ROs. Lipidation at C-terminal motifs (e.g., −CAAX) anchors nucleases to the host membranes which are used by the viruses to form ROs. Prenylated “CTIL”-motif targets proteins predominantly to endoplasmic reticulum membranes, while prenylated “CVIS”—to endo-lysosomal and plasma membranes.

FIG. 2 illustrates a schematic representation 200 of fusion between CRISPR-associated (Cas) proteins and guide RNA (gRNA) ribonucleoprotein (RNP) complexes, such as Cas13 or Cas7-11, and lipidation motifs (−CAAX). Different designs are used. Design 1—direct fusion between RNP and 4 amino acid motif (i.e., CTIL or CVIS), Design 2—fusion between RNP and CTIL motif through the peptide linkers of varying length (30-120 amino acids) derived from the C-terminal end of OAS1 human protein, Design 3—fusion between Cas protein and CVIL motif through peptide linkers of varying length (30-120 amino acids) derived from the C-terminal end of ZAP-L human protein, Design 4—fusion of Cas protein to CTIL or CVIL motifs through an artificial “flexible” linkers, which represents combinations of “GGS” motifs.

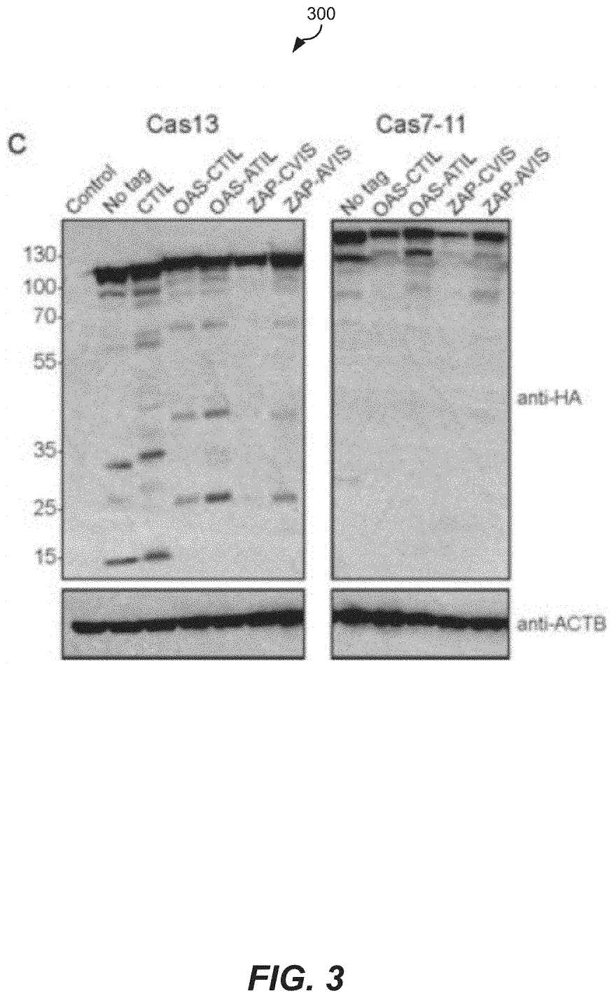

FIG. 3 is an image 300 of a Western blot of protein expression of Cas-CAAX fusions in HEK 293T cells was tested using Western Blot with anti-HA-tag antibodies. ACTB is used as a loading control; and

FIG. 4 illustrates an image 400 of fluorescent microscopy, demonstrating that proteins-of-interest fused to lipidation motifs change subcellular localization in HEK 293T cells. Bottom panel shows insets from the images on the top. Perinuclear localization characteristic of ER/Golgi association is indicated with white arrows.

CRISPR-Csm Cleavage and RNA Ligation Provides a Platform for Flexible and Robust RNA Editing

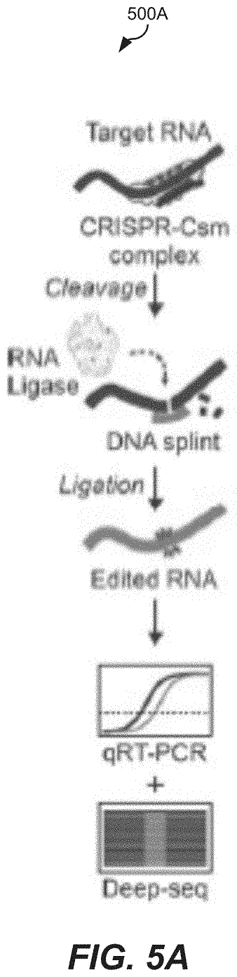

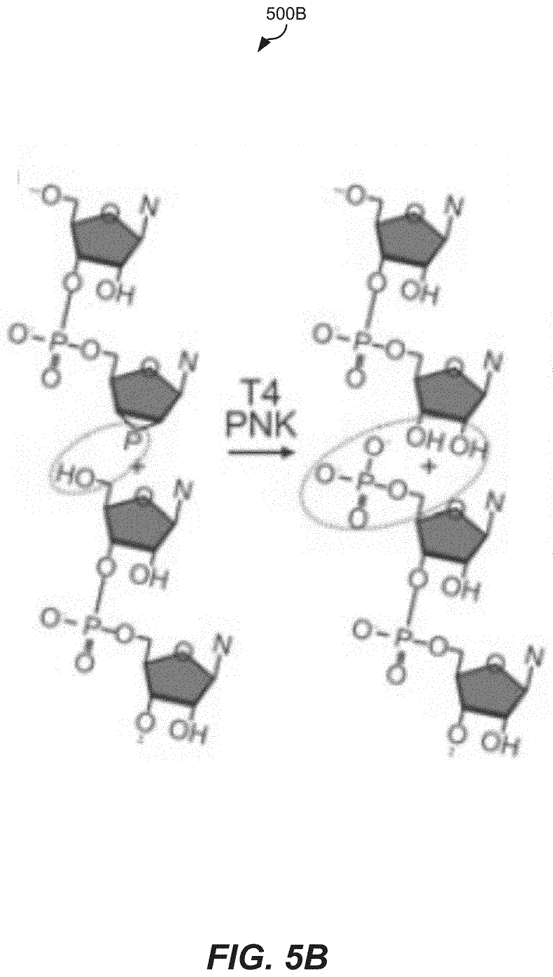

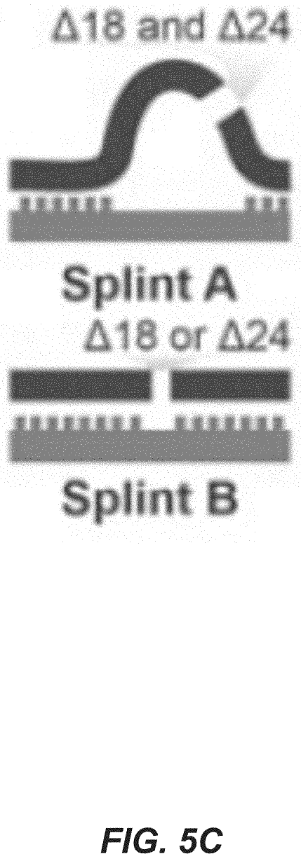

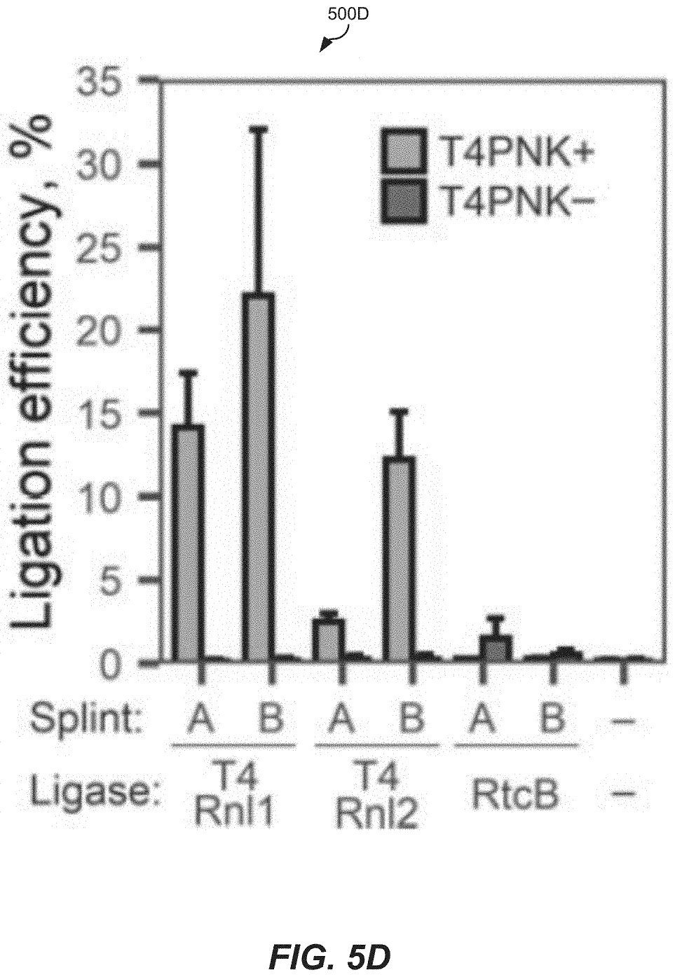

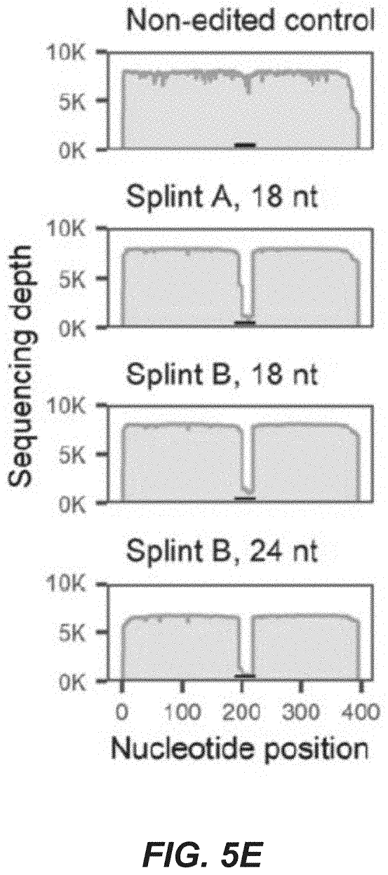

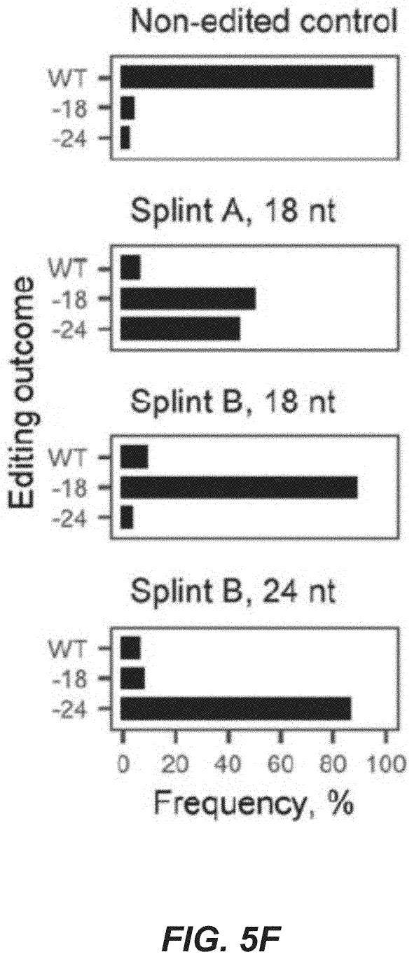

FIG. 5A illustrates a schematic diagram 500A showing RNA editing with type-III CRISPR systems and splinted ligation. Type-III CRISPR complexes generate multiple cuts in 6 nt intervals (red triangles) in target RNA. Type III complexes cut out a portion of target RNA and then resulting fragments are splint ligated to introduce edits.

FIG. 5B illustrates a schematic diagram 500B OF SthCsm-mediated cleavage. SthCsm-mediated cleavage generates 2′3′-cyclic phosphate and 5′-hydroxyl ends (left, substrate for RtcB ligase) that can be converted to 3′-hydroxyl and 5′-phosphate (right, substrate for T4 Rnl) using T4 polynucleotide kinase (PNK).

FIG. 5C illustrates a schematic diagram 500C showing examples of splint designs (splint A and splint B). Splint A imitates cuts in tRNA anti-codon loop, while splint B mimics nicked double-stranded RNA.

FIG. 5D illustrates a plot 500D of a comparison of RNA ligation efficiency. Plot 500D shows RNA ligation efficiency with combinations of splints shown in FIG. 5C and T4 RNA ligases (T4 Rnl) 1 and 2, and RtcB ligase. Ligation efficiency was measured by performing qRT-PCR across the cut site and quantifying signal relative to an uncut control (100%).

FIG. 5E illustrates plots of deep sequencing of RNA ligated using splint designs shown in FIG. 5C. Horizontal black bars in each of the plots show target site of the SthCsm complex.

FIG. 5F illustrates plots of quantification of editing outcomes shown in FIG. 5E.

CRISPR-Csm Cleavage and RNA Ligation Enables Programmed Deletions in RNA Virus Genome.

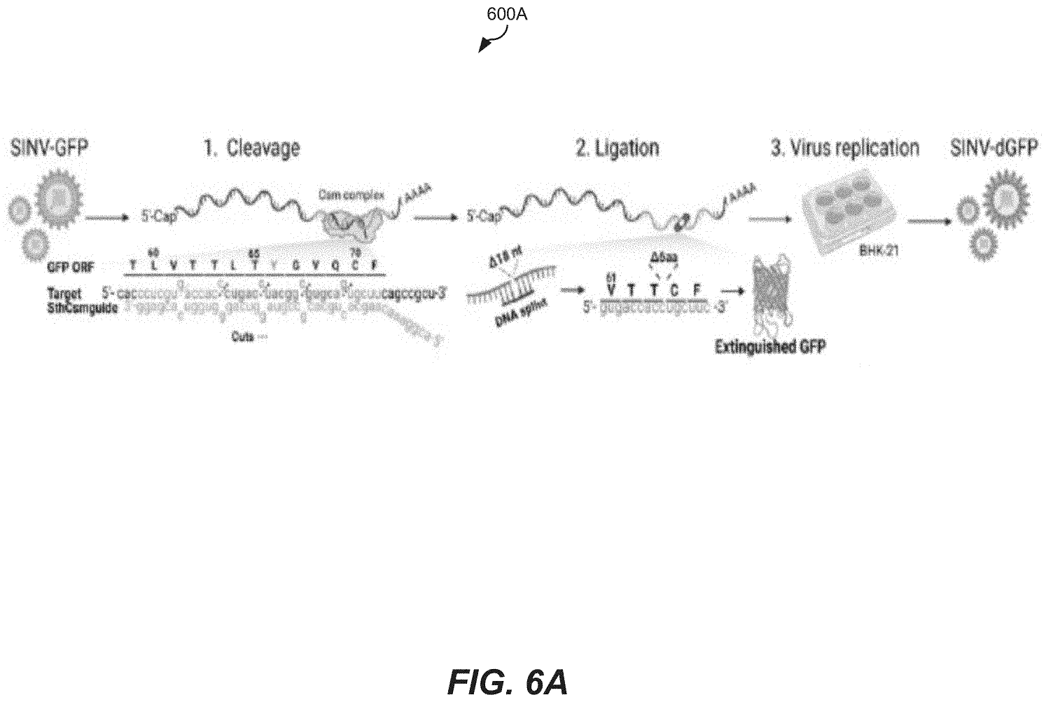

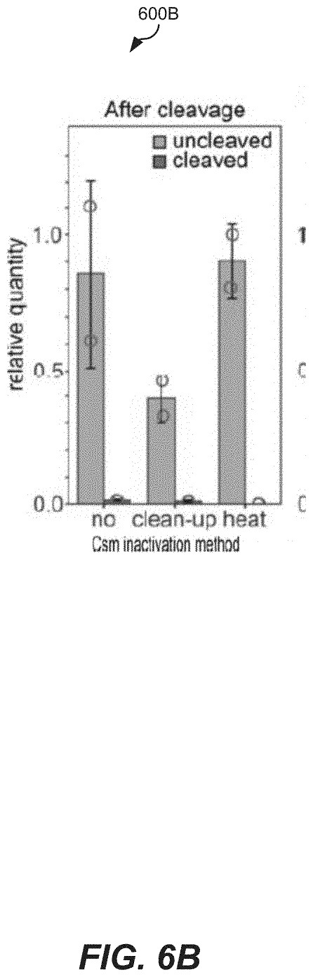

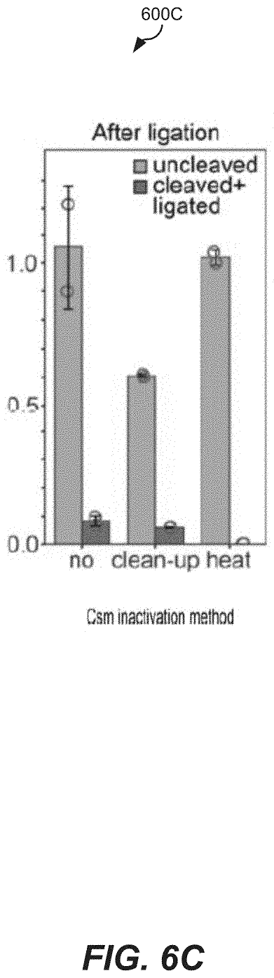

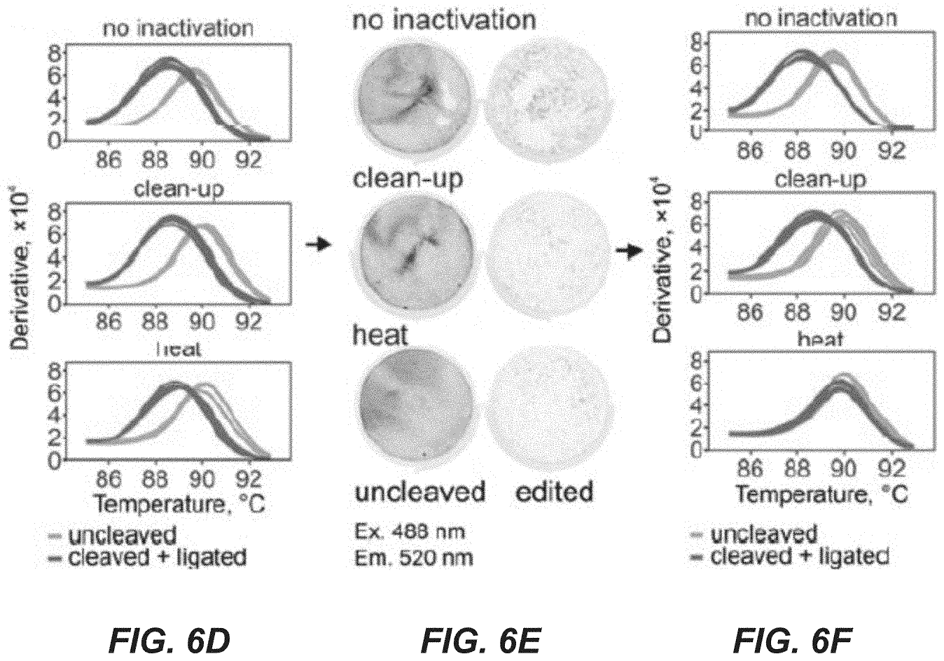

FIG. 6A illustrates a diagram 600 showing a pipeline for deleting 18 nucleotides in the GFP open reading frame (ORF) in the recombinant Sindbis-GFP (SINV-GFP) virus. The deletion eliminates 6 codons in the GFP gene including codons encoding amino acids (T65, Y66, G67) that form the chromophore in the fluorescent protein. Cleavage sites in the target RNA are shown with dotted diagonal lines Ends of RNA fragments are sequestered with complementary DNA splint and RNA is ligated. To recover edited virus, cells permissive to virus replication (i.e., BHK-21 cells) are transfected with the edited RNA. Edited virus encoding deletion mutant of the GFP gene (dGFP) is released in the supernatant of the transfected cells.

FIG. 6B illustrates a plot 600B of RNA aliquots collected after the cleavage with Csm complex.

FIG. 6C illustrates a plot 600C of RNA aliquots collected after and ligation with T4 RNA ligase. RNA aliquots in FIGS. 6B and 6C were reverse-transcribed and quantified with qPCR. Primers were designed to amplify cDNA across the target site. Relative quantities were calculated by normalizing to the uncut RNA control. CRISPR-Csm after the cleavage was either removed using a column-based RNA purification (“clean-up”), inactivated with heat (“heat”), or carried over to the next reaction without inactivation (“no”).

FIG. 6D illustrates a melting curve analysis of the qPCR products in FIG. 6C. Peaks indicate melting temperature of the generated products. Edited RNA with 18 nt deleted produces shorter qPCR products with decreased melting temperature.

FIG. 6E illustrates images of BHK-21 cells that were transfected with edited RNA of the SINV-GFP and imaged 24 hour later using Typhoon Imager with Cy2 setting (ex. 488/em. 520) to capture GFP signal. Edited virus is expected to produce no GFP signal.

FIG. 6F illustrates melting curve analysis of the RT-qPCR products that were generated with RNA extracted from supernatants of BHK-21 cells 24 hours post transfection. Melting profiles of the qPCR product with edited sample indicate successful deletion in the target site.

CRISPR-Csm Cleavage and RNA Ligation Enables Programmed Substitutions in RNA Virus Genome.

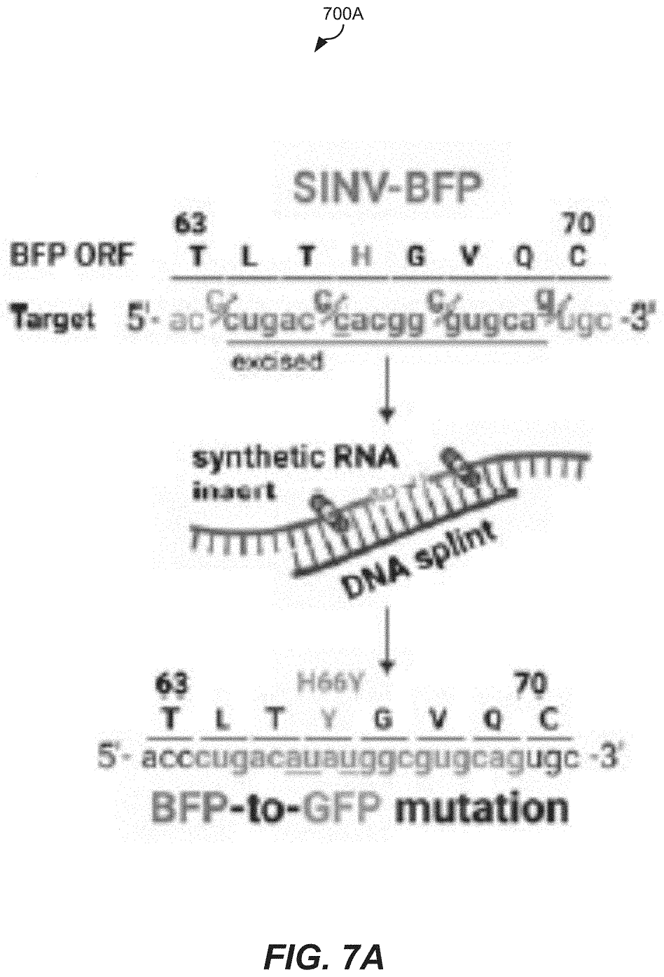

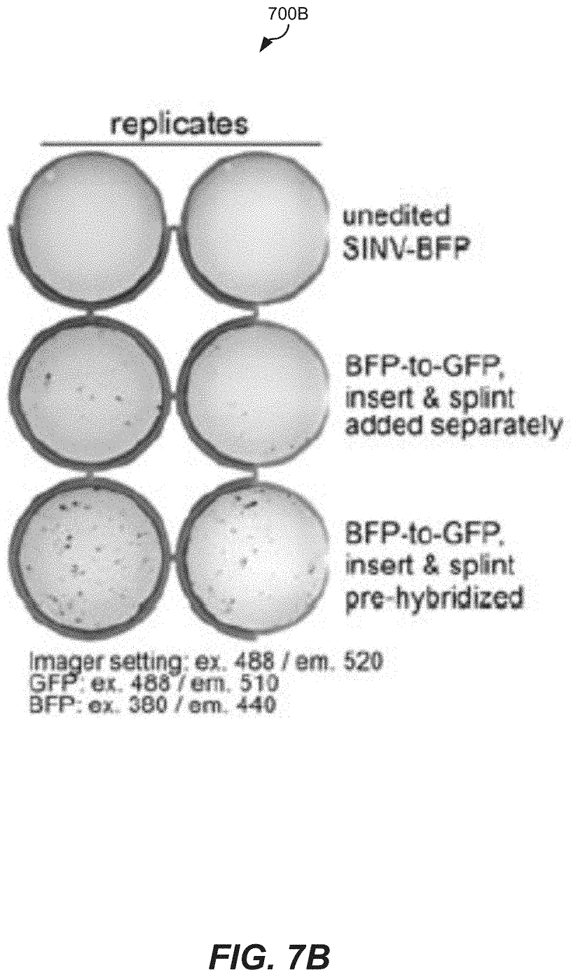

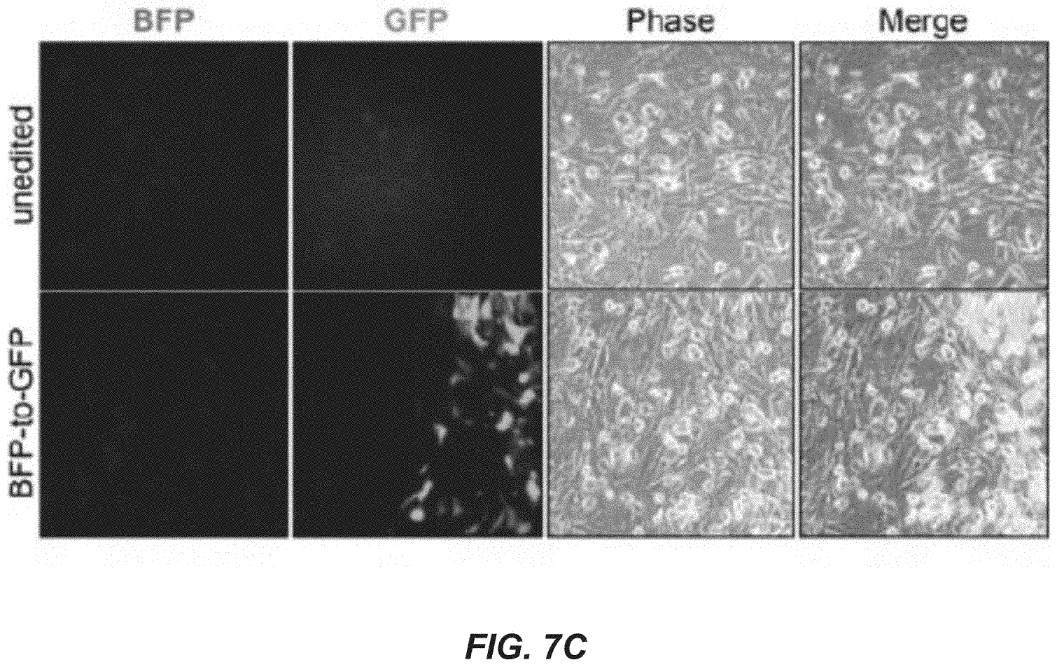

FIG. 7A illustrates a diagram 700A showing a pipeline for substituting 18 nucleotides in the BFP open reading frame (ORF) in the recombinant Sindbis-BFP (SINV-BFP) virus. CRISPR-Csm cleavage sites are shown with diagonal dotted lines. Excised sequence is underlined. DNA fragments are sequestered with a complementary DNA splint. Synthetic RNA oligonucleotide complementary to the splint is inserted in the viral RNA to substitute excised sequence. The C>U substitution changes histidine residue in position 66 (H66) to tyrosine (H66Y mutation), which in turn changes the fluorescence from blue to green spectrum (BFP-to-GFP). Additional silent substitutions (underlined) were added to distinguish edited RNA from contamination with the SINV-GFP RNA (see FIG. 6A).

FIG. 7B illustrates images of BHK-21 cells transfected with the edited viral RNA were imaged after 24 hours incubation using Typhoon Imager with the Cy2 setting (ex. 488 nm/em. 520 nm). This laser & filter setting does not detect cells replicating unedited SINV-BFP (ex. 380/em. 440), while cells with edited BFP-to-GFP virus are visible.

FIG. 7C illustrates images of transfected BHK-21 cells shown in FIG. 7B imaged with fluorescent microscope at 20× magnification.

Plaque-Purification and Nanopore Sequencing of Edited Viral Clones

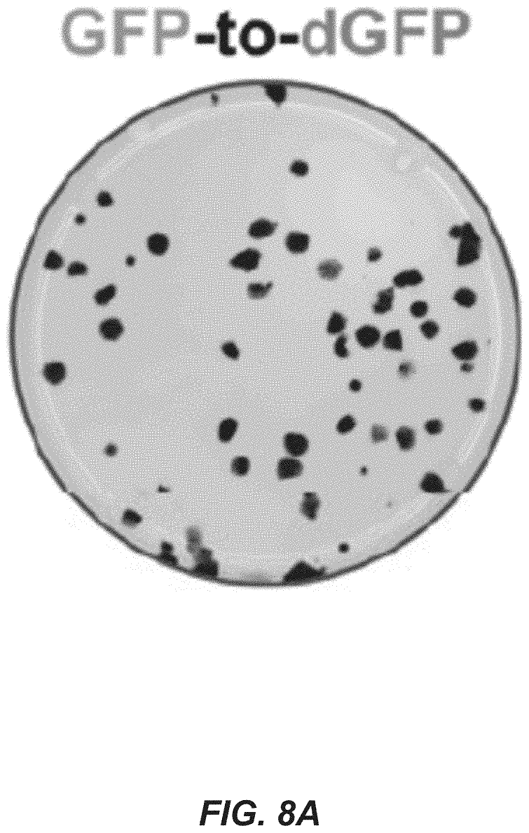

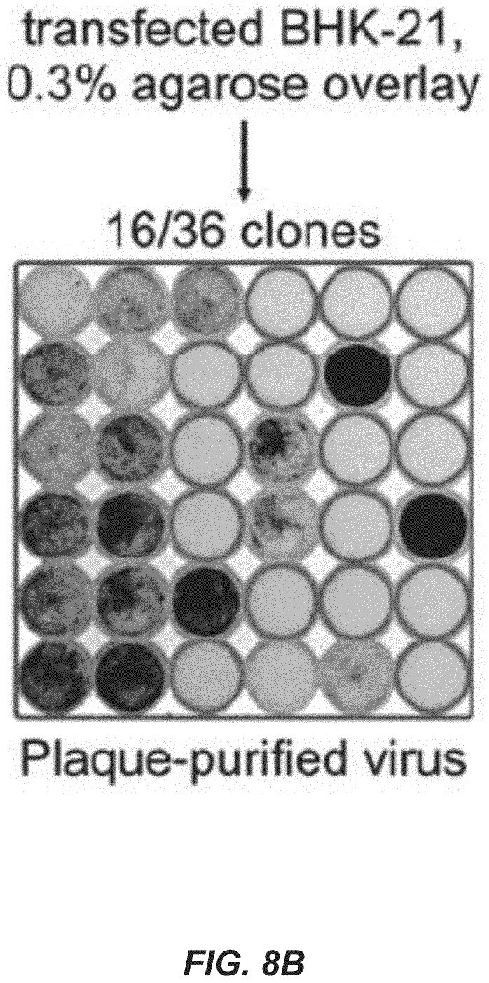

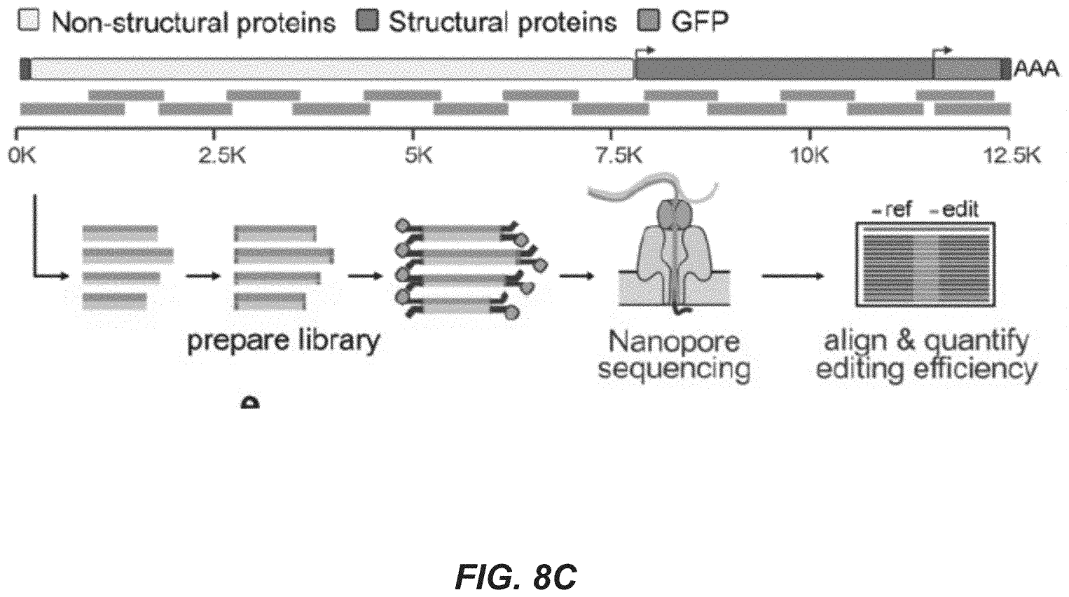

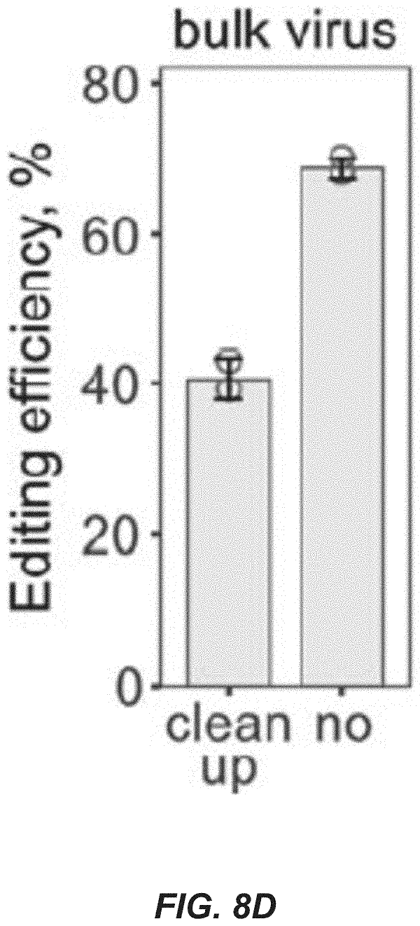

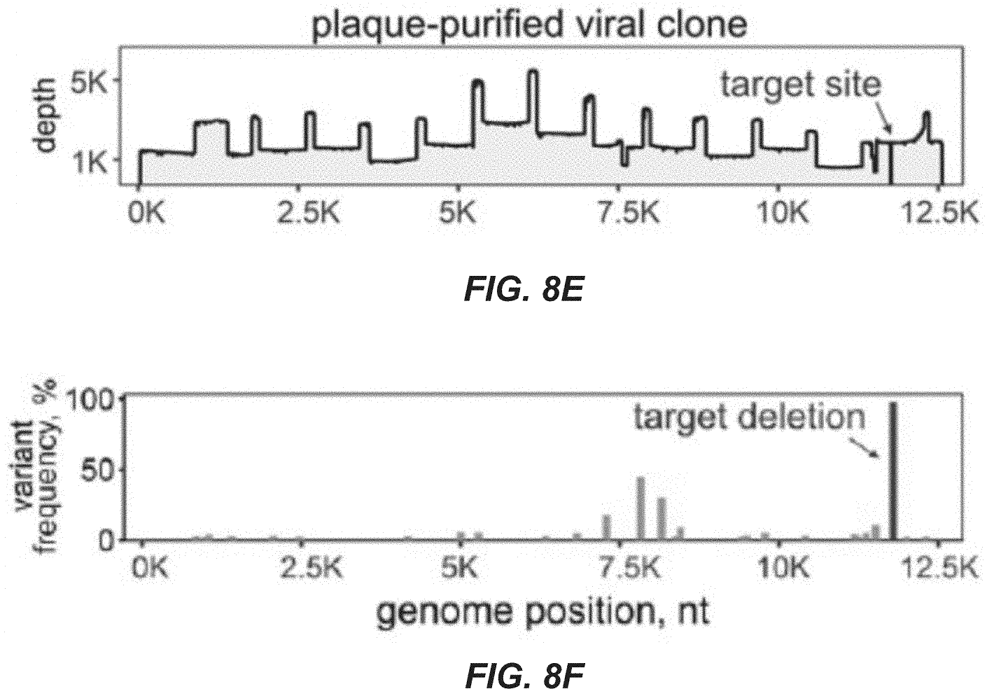

FIG. 8A illustrates imaging of genomic RNA of Sindbis-GFP virus (SINV-GFP), which was edited to delete codons encoding for the chromophore (T65, Y66, G67) in the GFP gene. Edited RNA was transfected in BHK-21 cells. Transfected cells were seeded on a monolayer of BHK-21 in 100 mm petri dishes. After transfected cells attached to the surface, supernatants were removed and DMEM media supplemented 0.3% agarose was overlayed. Viral plaques were allowed to form for 48 hours and imaged using Typhoon Imager with Cy2 setting (ex. 488/em. 520) to capture GFP signal. Edited virus is expected to produce no GFP signal, while cells infected with residual unedited virus will be visible.

FIG. 8B illustrates images of viral clones (n=36) that were collected from plaques and transferred into 48-well plate with BHK-21 cells. The next day, cells were visualized using Typhoon Imager with Cy2 setting (ex. 488/em. 520). Edited virus is expected to produce no GFP signal, therefore supernatants from wells that demonstrate cytopathic effect and no GFP signal (n=16, marked with red circles) were collected for RNA extraction.

FIG. 8C illustrates a schematic diagram of an SINV-GFP genome. RNA extracted from supernatant of infected cells was reverse transcribed and amplified to produce overlapping amplicons that span the entire viral genome (horizontal turquoise bars). Further, amplicons were used to prepare sequencing libraries, which included end repair, barcode ligation, and sequencing adapter ligation (bottom). Resulting libraries were sequenced with Oxford Nanopore technology. Sequencing reads were aligned to a reference genome sequence and editing outcomes were quantified.

FIG. 8D illustrates a plot of frequency of programmed edits in the bulk virus population before the plaque purification. In the editing process CRISPR-Csm complex was removed after the cleavage step (“Clean up”), or was carried over to the ligation reaction without inactivation (“no”)(see FIG. 2b,e for details).

FIG. 8E illustrates a representative sequencing depth plot for a plaque-purified viral clone in FIG. 8B. Arrow points to a drop in read depth at the targeted site, which indicates a deletion in the viral genome. f, Sequencing reads were compared to a reference and frequency of sequence variants was quantified. Red bar shows frequency of the programmed deletion at the target site (98.1%), turquoise bars show single nucleotide variants resulting from viral intra-host diversity.

The term “engineered”, and similar terms may refer to a deliberate generation of a system that is otherwise non-naturally occurring. Such engineering may include introducing one or more mutations to a genetic sequence, designing a genetic sequence, combining a set of components such as proteins and detection components where such combination does not occur in nature, and/or otherwise generating a non-naturally occurring system to edit nucleic acid such as RNA.

The engineered type III CRISPR complex may include a nuclease that cleaves RNA at specific sites guided by a CRISPR RNA (crRNA) sequence, which may be programmable. The term “programmatic”, “programmable”, and similar terms may refer to modifying a CRISPR complex or components thereof. An example of programmable delivery of RNA-guided CRISPR-Cas effectors (such as CRISPR-Cas proteins) may include fusing one or more lipidation motifs to a CRISPR nuclease to anchor the CRISPR nuclease to membranes appropriated by RNA viruses.

In some instances, a programmable crRNA sequence can be designed to guide the engineered type III CRISPR complex to a specific target portion of interest in the RNA. For example, sequence specific targeting of crRNAs may be performed by designing synthetic spacer sequences. The synthetic spacer sequences may be between 20 and 60 nucleotides long that separate the repeat sequences or end with a self-cleaving ribozyme, such that the crRNA is processed into a short (20-100 nt) crRNA that is incorporated into an assembly of one or more Cas proteins, which together form a ribonucleoprotein complex that stably binds and cleaves RNAs that are complementary to the guide (spacer) sequence. The spacer sequences are designed to be complementary to a target sequence and intentionally designed to avoid complementarity to other “non-target” RNAs. In some examples, crRNA-guides are designed to include a protospacer flanking sequence (PFS) that facilitates binding, cleavage, or cyclic nucleotide synthesis. In other examples, the PFS is any sequence that is not complementary to the 5′ repeat sequence of the crRNA. The programmable crRNA sequence may therefore facilitate specific cleavage of the RNA at one or more specific sites, enabling programmatic RNA editing. One example of an engineered type III CRISPR complex that may be used is a Csm complex, which is a type III-A CRISPR complex that has RNase activity. The Csm complex may be a SthCsm, derived from S. thermophilus.

Various examples described herein will refer to treatment involving a viral RNA genome. However, any cellular location may be targeted based on the disclosures herein. Target RNA or other target may be obtained from a subject. For example, the target may be an RNA of an organism that infects a host organism. In particular, the target RNA may be an RNA genome of a virus that has infected the subject. In another example, the target RNA may be the RNA of the subject. A subject may refer to an animal, such as a mammalian species (preferably human) or avian (e.g., bird) species, or other organism, such as a plant. More specifically, a subject can be a vertebrate, e.g., a mammal such as a mouse, a primate, a simian or a human. Animals include farm animals, sport animals, and pets. A subject can be a healthy individual, an individual that has symptoms or signs or is suspected of having a disease or a predisposition to the disease, or an individual that is in need of therapy or suspected of needing therapy.

A genetic modification or mutation in the context of an engineered system may refer to an alteration, variant or polymorphism in a nucleic acid that may result in altered or disabled functionality of a corresponding protein. Such alteration, variant or polymorphism can be with respect to a reference genome, the subject or other individual. Variations include one or more single nucleotide variations (SNVs), insertions, deletions, repeats, small insertions, small deletions, small repeats, structural variant junctions, variable length tandem repeats, and/or flanking sequences, CNVs, transversions, gene fusions and other rearrangements may also be considered forms of genetic variation. A variation can be a base change, insertion, deletion, repeat, copy number variation, transversion, or a combination thereof.

A “polynucleotide”, “nucleic acid”, “nucleic acid molecule”, or “oligonucleotide” may each refer to a polymer of nucleosides (including deoxyribonucleosides, ribonucleosides, or analogs thereof) joined by inter-nucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “AUGCCUG,” it will be understood that the nucleotides are in 5′ to 3′ order from left to right and that “A” denotes adenosine, “C” denotes cytidine, “G” denotes guanosine, and “U” denotes uracil, unless otherwise noted. The letters A, C, G, and U (or “T” denoting thymine in DNA) may be used to refer to the bases themselves, to nucleosides, or to nucleotides comprising the bases, as is standard in the art.

All patent filings, websites, other publications, sequence listings, accession numbers and the like cited above or below are incorporated by reference in their entirety for all purposes to the same extent as if each individual item were specifically and individually indicated to be so incorporated by reference.

If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

What is claimed is:

1. A method of programmable delivery of Clustered Regularly Interspaced Short Palindromic Repeats (CRISPR)-Cas proteins for subcellular localization, the method comprising:
fusing one or more lipidation motifs to a CRISPR-Cas protein to generate an engineered CRISPR protein, wherein the one or more lipidation motifs are post-translationally modified to anchor the one or more lipidation motifs and the fused CRISPR-Cas protein to a target cellular location; and
administering the engineered CRISPR protein to a subject.

2. The method of claim 1, wherein the cellular location is a membrane of a subcellular organelle.

3. The method of claim 1, wherein the CRISPR-Cas protein is a CRISPR-Cas nuclease that cleaves target nucleic acid as part of a treatment regimen for the subject.

4. The method of claim 3, wherein the target nucleic acid is viral nucleic acid.

5. The method of claim 3, wherein the cellular location reduces cellular toxicity of the CRISPR-Cas nuclease.

6. The method of claim 1, wherein the one or more lipidation motifs comprise a CTIL motif.

7. The method of claim 1, wherein the one or more lipidation motifs comprise a CVIS motif.

8. The method of claim 1, wherein fusing the one or more lipidation motifs to the CRISPR-Cas protein comprises inserting a linker to account for amino acids that affect the efficiency of the post-translational modification.

9. The method of claim 8, wherein the linker is derived from a C-terminal end of OAS1 or ZAP-L proteins.

10. An engineered Clustered Regularly Interspaced Short Palindromic Repeats (CRISPR)-Cas protein, comprising:
a CRISPR-Cas protein; and
one or more lipidation motifs fused to the CRISPR-Cas protein, the one or more lipidation motifs being post-translationally modified to anchor the one or more lipidation motifs and the fused CRISPR-Cas protein to a target cellular location.

11. The engineered CRISPR-Cas protein of claim 10, further comprising:
a linker to account for amino acids that affect the efficiency of the post-translational modification.

12. A method of generating an engineered Clustered Regularly Interspaced Short Palindromic Repeats (CRISPR)-Cas protein, comprising:
generating a library of CRISPR-Cas proteins comprising a CRISPR-Cas protein; and
contacting one or more lipidation motifs with the library CRISPR-Cas proteins to fuse the one or more of lipidation motifs to the CRISPR-Cas protein, wherein the one or more lipidation motifs are post-translationally modified to anchor the one or more lipidation motifs and the fused CRISPR-Cas protein to a target cellular location.
